(19) United States
(12) Patent Application Publication
(10) Pub. No.: US 2008/0194709 A1
(43) Pub. Date: Aug. 14, 2008

(54) Dried Emulsion, Method For Preparing Same and Uses Thereof

(75) Inventor: Jean-Pierre Hecaen, Stains (FR)

Correspondence Address: BUCHANAN, INGERSOLL & ROONEY PC, POST OFFICE BOX 1404, ALEXANDRIA, VA, 22313-1404, US

(73) Assignee: Rhodia Chimie, Aubervilliers (FR)

(21) Appl. No.: 11/791,224

(22) PCT Filed: Nov. 22, 2005
(86) PCT No.: PCT/FR05/02897

§ 371 Date: Dec. 5, 2007

(30) Foreign Application Priority Data
Nov. 22, 2004 (FR) 0412366

(51) Int. Cl. A61K 47/32; C08G 63/00; C08L 31/02; C08L 37/00; C08L 5/00; A01P 15/00; A61Q 99/00; C08K 5/10; C08K 5/09; C11D 3/37

(52) U.S. Cl. 514/772.6; 524/599; 524/556; 524/549; 524/27; 524/315; 524/300; 510/361; 510/230

(57) ABSTRACT

Dried emulsions suited for formulation into, e.g., plant protection and detergent compositions comprise a matrix of a water-soluble or water-dispersible polymer, in which is dispersed a hydrophobic phase, and also containing a non-polyalkoxylated surfactant, the water-soluble or water-dispersible polymer being a polycarboxylate including hydrophobic units.

The present invention relates to a dried emulsion comprising a matrix comprising a water-soluble or water-dispersible polymer, in which is dispersed a hydrophobic phase. The invention also relates to a process for preparing the dried emulsion, and to uses of said emulsion.

Dried emulsions are compositions comprising a liquid hydrophobic phase dispersed in a solid matrix. They may be obtained by drying an emulsion comprising an aqueous phase in which is dispersed the liquid hydrophobic phase, generally by means of a surfactant, the aqueous phase comprising the water-soluble or water-dispersible compound that will constitute all or part of the matrix after drying. Compositions in the form of dried emulsions and processes for preparing them are known to those skilled in the art. Dried emulsions are generally in the form of powder or granules.

Dried emulsions comprising a polyalkoxylated surfactant and a matrix comprising a polymer comprising hydrophilic units and units comprising hydrophobic groups or grafts are thus known. Such dried emulsions are described, for example, in documents WO 00/26280 (R 98145), WO 02/32563 (R 00137) and WO 03/006148 (R 01103).

Dried emulsions are generally intended to be dispersed in an aqueous composition to obtain an emulsion or a dispersion comprising the hydrophobic phase dispersed in the aqueous composition. Dried emulsions may thus facilitate or make economically viable the handling and/or transportation and/or vectorization and/or protection of the hydrophobic phase. Dried emulsions may thus be formulated with other solid compounds and be placed in contact with water by the final user. It is thus possible, for example, to prepare powder mixtures. This is the case, for example, for certain plant-protection compositions, and for detergent formulations in powder or granule form. Dried emulsions may also be placed in contact with water by an operator preparing an aqueous formulation comprising the hydrophobic phase. It is also noted that it may be advantageous to initiate the redispersion by an external factor (for example a change in pH, in temperature, in chemical composition of the environment, by the release of aqueous substances such as urine or sweat, etc.) or to control the redispersion kinetics.

Qualities of a dried emulsion include: easy redispersion in water, no coalescence of the water-immiscible hydrophobic phase during the drying and/or redispersion, good flowability of powder, an easily manipulable form, for example a non-oily form, control of coalescence or of a size of droplets of the hydrophobic phase before drying or after drying, on redispersion.

Novel dried emulsions that may have improved qualities among those mentioned above have now been found.

Thus, the invention proposes a dried emulsion comprising a matrix comprising a water-soluble or water-dispersible polymer, in which is dispersed a hydrophobic phase, characterized in that:
the dried emulsion comprises a non-polyalkoxylated surfactant,
the water-soluble or water-dispersible polymer included in the matrix is a polycarboxylate comprising hydrophobic units.

The invention also proposes a process for preparing such emulsions, and uses thereof.

The invention also proposes a mixture of surfactants that is particularly suitable for the use of the dried emulsions according to the invention, or even for the use of emulsions in general.

### DEFINITIONS

In the present patent application, the term water-soluble or water-dispersible polymer means a polymer which, at a concentration of 10% by weight in water, at a temperature of 25° C., shows no macroscopic phase separation. In the present patent application, the water-soluble or water-dispersible quality is considered at the pH of preparation of the dried emulsion and/or at the pH of use of the dried emulsion during redispersion.

In the present patent application, the term hydrophobic phase means a compound or a composition comprising several compounds, which is water-immiscible (forming a macroscopic separation of phases), at a concentration of 10% by weight, at a temperature of 25° C. In the present patent application, the hydrophobic or water-dispersible phase quality is considered at the pH of preparation of the dried emulsion and/or at the pH of use of the dried emulsion during redispersion.

In the present patent application, unless otherwise mentioned, the amounts of ingredients and the ratios are given by weight of solids.

### Ingredients of the Dried Emulsion

The dried emulsion comprises a matrix comprising a water-soluble or water-dispersible polymer, in which is dispersed a hydrophobic phase. The hydrophobic phase may be liquid or solid. It is preferably liquid. When the hydrophobic phase is a solid phase, it may have been liquefied by heating and emulsified during the preparation of the emulsion of the dried emulsion. The hydrophobic phase is present in the form of inclusions (droplets if it is a liquid phase) in the matrix, advantageously with a mean size of between 0.1 and 50 μm and preferably between 1 and 10 μm, for example between 1 and 5 μm (determined using a Horiba laser scattering granulometer). The mean size may vary according to the final application.

Various ingredients that may be included in the composition of the dried emulsion are detailed below.

### Hydrophobic Phase

The hydrophobic phase may comprise compounds of any kind, alone or as mixtures, optionally dissolved or dispersed in a hydrophobic solvent. Needless to say, the hydrophobic phase can consist merely of a simple hydrophobic liquid. The hydrophobic phase may be an active hydrophobic compound, or a mixture comprising active hydrophobic compound dissolved or dispersed in a hydrophobic solvent.

The following are given as examples of hydrophobic phase or of compounds included in the hydrophobic phase:
silicones, for example silicone oils and gums, of MD, MTD or MQ type, optionally dissolved in solvents, and optionally functionalized with groups such as amines, alcohols, polyols, etc. Such silicones are known to those skilled in the art;
fragrances;
organic, mineral or plant or mineral oils, and derivatives of these oils, said oils and derivatives being water-immiscible;
water-immiscible organic solvents;
water-insoluble or water-indispersible active materials, optionally dissolved in a solvent;
mixtures thereof, as solutions, dispersions or emulsions.

In the field of agrochemistry, the plant-protection active materials may be chosen from the family of α-cyano-phenoxybenzylcarboxylates or α-cyano-halo-phenoxy-carboxylates, the family of N-methylcarbonates comprising aromatic substituents, and active materials such as Aldrin, Azinphos-methyl, Benfluralin, Bifenthrin, Chlorphoxim, Chlorpyrifos, Fluchloralin, Fluoroxypyr, Dichloruos, Malathion, Molinate, Parathion, Permethrin, Profenofos, Propiconazole, Prothiofos, Pyrifenox, Butachlor, Metolachlor, Chlorimephos, Diazinon, Fluazifop-P-butyl, Heptopargil, Mecarbam, Propargite, Prosulfocarb, Bromophos-ethyl, Carbophenothion, Cyhalothrin, Novaluron, Deltamethrine, Pendimethalin, Fluquinconazole, Tebuconazole, Alphamethrine, Chlothianidine, Betacyfluthrine, Cyfluthrine, Lambda-Cyhalothrine, Cyhalothrine, Fipronil, Thiaclopride, Imidaclopride, Phenmediphame, Desmediphame, Amidosulfuron, Ethofumesate. The plant-protection active materials may be used in the presence of standard additives chosen, for example, from adjuvants for increasing the efficacy of the active material, antifoams, anticaking agents and fillers, which may be water-soluble or water-insoluble.

These active materials may be dissolved in a hydrophobic solvent chosen, for example, from aromatic hydrocarbon-based solvents such as xylene, dibenzyltoluene, phosphate or phosphonate solvents such as tri-n-butyl phosphate (TBP), aliphatic hydrocarbon-based solvents such as mineral or plant oils, alcohols such as cyclohexanol, ketones such as cyclohexanone, amides such as N,N-dimethylcaprylamide-capramide, pyrrolidones such as N-methylpyrrolidone, N-octylpyrrolidone, N-dodecylpyrrolidone or N-cyclohexylpyrrolidone or alkyllactates, that dissolve them.

The hydrophobic phase may especially be a hydrophobic phase comprising deltamethrin and tri-n-butyl phosphate (TBP). However, it may be different than a hydrophobic phase comprising both deltamethrin and TBP. The hydrophobic phase may especially be a hydrophobic phase comprising deltamethrin and a xylene-based solvent such as Solvesso 200 or 200 ND. However, it may be different than a hydrophobic phase comprising both deltamethrin and a xylene-based solvent such as Solvesso 200 or 200 ND. The hydrophobic phase may especially be a hydrophobic phase comprising deltamethrin, a xylene-based solvent such as Solvesso 200 or 200 ND and tri-n-butyl phosphate (TBP). However, it may be different than a hydrophobic phase comprising both deltamethrin, a xylene-based solvent such as Solvesso 200 or 200 ND, and TBP.

Similarly, as active materials that are suitable in the field of plant-protection formulations, mention may be made of plant oils, mineral oils, silicone oils, silicone antifoams, etc.

As examples of active materials that may be used in the cosmetics field, mention may be made of silicone oils belonging, for example, to the dimethicone family; lipophilic vitamins, for instance vitamin A and its derivatives, vitamin B2, pantothenic acid, vitamin D and vitamin E; monoglycerides, diglycerides and triglycerides; fragrances; bactericides; UV absorbers, for instance aminobenzoate derivatives of PABA and PARA type, salicylates, cinnamates, anthranilates, dibenzoylmethanes and camphor derivatives, and mixtures thereof.

Anti-aging agents may similarly be used. Examples of such agents that may especially be mentioned include retinoids, α- and β-hydroxy acids, salts thereof and esters thereof, liposoluble vitamins, ascorbyl palmitate, ceramides, pseudoceramides, phospholipids, fatty acids, fatty alcohols, cholesterol and sterols, and mixtures thereof. As preferred fatty acids and fatty alcohols, mention may be made more particularly of those with linear or branched alkyl chains containing from 12 to 20 carbon atoms. This may especially be linoleic acid.

It is similarly possible to use anticellulite agents, especially such as isobutylmethylxanthine and theophylline; and also antiacne agents, for instance resorcinol, resorcinyl acetate, benzoyl peroxide and numerous natural compounds.

Flavorings, fragrances, essential oils and essences may also be used as hydrophobic active material. Examples that may be mentioned include oils and/or essences of mint, of spearmint, of peppermint, of menthol, of vanilla, of cinnamon, of laurel, of aniseed, of eucalyptus, of thyme, of sage, of cedar leaf, of nutmeg, of citrus fruit (lemon, lime, grapefruit or orange) or of fruits (apple, pear, peach, cherry, plum, strawberry, raspberry, apricot, pineapple, grape, etc.), alone or as mixtures.

The antimicrobial agents may be chosen from thymol, menthol, triclosan, 4-hexylresorcinol, phenol, eucalyptol, benzoic acid, benzoyl peroxide and butyl paraben, and mixtures thereof.

As examples of active materials that are suitable for the invention and that may be used in the field of paints, mention may be made of alkyd resins, epoxy resins and masked or unmasked isocyanates.

In the paper sector, mention may be made, inter alia, of resins such as alkylketene dimer (AKD) or alkenyl-succinic anhydride (ASA).

In the field of detergency, possible active materials that may be mentioned include amino silicones as softeners, silicone antifoams, antimicrobial agents, fragrances, oils and essences, etc. In this respect, reference may be made to the list of compounds of this type that are indicated in the context of active materials for the cosmetics field.

Among the suitable hydrophobic active materials, mention may also be made of:
organic oils/fats/waxes of animal origin or of plant origin;
mineral oils/waxes;
products derived from the alcoholysis of the abovementioned oils;
mono-, di- and triglycerides;
saturated or unsaturated fatty acids containing 10 to 40 carbon atoms; esters of such acids and of alcohols containing 1 to 6 carbon atoms;
saturated or unsaturated monoalcohols containing 8 to 40 carbon atoms;
   these compounds being used alone or as a mixture.

As organic oils/fats/waxes of animal origin, mention may be made, inter alia, of sperm whale oil, whale oil, seal oil, shark oil, cod liver oil, pig fat, sheep fat (tallow), perhydrosqualene and beeswax, alone or as a mixture.

As examples of organic oils/fats/waxes of plant origin, mention may be made, inter alia, of rapeseed oil, sunflower oil, groundnut oil, olive oil, walnut oil, corn oil, soybean oil, avocado oil, linseed oil, hemp oil, grapeseed oil, coconut oil, palm oil, cottonseed oil, palm kernel oil, babassu oil, jojoba oil, sesameseed oil, castor oil, macadamia oil, sweet almond oil, carnauba wax, shea butter, cocoa butter and peanut butter, alone or as a mixture.

As regards the mineral oils/waxes, mention may be made, inter alia, of naphthenic oils, paraffinic oils (petroleum jelly), isoparaffinic oils and paraffinic waxes, alone or as a mixture.

Products derived from the alcoholysis of the above-mentioned oils may also be used.

As regards the fatty acids, they are saturated or unsaturated, contain 10 to 40 carbon atoms and more particularly 18 to 40 carbon atoms, and may comprise one or more conjugated or nonconjugated ethylenic unsaturations. It should be noted that said acids may comprise one or more hydroxyl groups.

Examples of saturated fatty acids that may be mentioned include palmitic acid, stearic acid, isostearic acid and behenic acid.

Examples of unsaturated fatty acids that may be mentioned include myristoleic acid, palmitoleic acid, oleic acid, erucic acid, linoleic acid, linolenic acid, arachidonic acid and ricinoleic acid, and also mixtures thereof.

As regards fatty acid esters, mention may be made of esters of the acids listed above, for which the part derived from the alcohol contains 1 to 6 carbon atoms, for instance methyl, ethyl, propyl, isopropyl, etc. esters.

It is mentioned that the hydrophobic phase may comprise a water-immiscible intermediate phase in which is dispersed an inner phase that is immiscible or insoluble in the intermediate phase. In this case, the system may be referred to as a dried multiple emulsion.

Needless to say, it is not excluded for the dried emulsion to comprise several different hydrophobic phases dispersed the matrix, consisting two populations of inclusions.

### Water-Soluble or Water-Dispersible Polymer

The water-soluble or water-dispersible polymer of the matrix is a polycarboxylate comprising hydrophobic units.

Advantageously, it is a copolymer comprising units derived from the following monomers:
(I) monocarboxylic or polycarboxylic acid, or linear or branched, ethylenically unsaturated aliphatic, cyclic or aromatic anhydride,
(II) monomer of formula (R²)(R²)C═CH(R³) in which
   R³ is a hydrogen atom or a methyl group, and
   R², which may be identical or different, represent a hydrogen atom or a linear or branched, optionally cyclic, C₂-C₁₀ saturated or ethylenically unsaturated aliphatic or aromatic radical, on condition that the two radicals R² are not hydrogen atoms, and
optionally (III) polyoxyalkylenated ester of an ethylenically unsaturated carboxylic acid.

The monomer (I) advantageously has the formula:

(R³)HC═C(R¹)—COOX (Ia)

in which:
R³ is a hydrogen atom or a methyl group,
R¹ is a hydrogen atom, a C₁-C₁₀ hydrocarbon-based radical optionally comprising a group —COOX, or a group —COOX, optionally forming with the group —COOX an anhydride group —CO—O—OC—, and
X is a hydrogen atom or a cation, for example the sodium or potassium cation, or the ammonium cation.

For example, the monomer (I) is advantageously chosen from acrylic, methacrylic, citraconic, maleic, fumaric, itaconic or crotonic acid or anhydride, in acid form or in the form of salts, for example in the form of the sodium or potassium salt.

Advantageously, the monomer (II) is chosen from 1-butene, isobutylene, n−1-pentene, 2-methyl-1-butene, n−1-hexene, 2-methyl-1-pentene, 4-methyl-1-pentene, 2-ethyl-1-butene, diisobutylene (or 2,4,4-trimethyl-1-pentene) and 2-methyl-3,3-dimethyl-1-pentene.

The monomer (III) may correspond to the following formula:

CH₂═C(R³)—C(O)—O—[CH₂CH(R⁴)O]ₘ—[CH(R⁵)—CH₂O]ₙ—R⁶
in which formula:
R³ is a hydrogen atom or a methyl radical,
R⁴ and R⁵, which may be identical or different, represent a hydrogen atom or an alkyl radical containing from 1 to 4 carbon atoms,
R⁶ is an alkyl, aryl, alkylaryl or arylalkyl radical containing from 1 to 30 and preferably from 8 to 30 carbon atoms,
n is between 2 and 100 and preferably between 6 and 100,
m is between 0 and 50,
   with the proviso that n is greater than or equal to m and their sum is between 2 and 100 and preferably between 6 and 100.

Preferably, monomers of formula (III) are used for which R⁶ is an alkyl radical containing from 8 to 30 carbon atoms, or a phenyl radical substituted with one to three 1-phenylethyl groups, or an alkylphenyl radical whose alkyl radical contains from 8 to 16 carbon atoms.

Among the monomers of this type that may be used, mention may be made of those described in patents EP 705 854, U.S. Pat. No. 4,138,381 or U.S. Pat. No. 4,384,096.

The copolymer may also comprise units derived from an optional monomer (IV) chosen from the following monomers:
vinylaromatic monomers, for instance styrene or vinyltoluene,
C₁-C₂₀ alkyl esters of ethylenically unsaturated acids, for instance methyl, ethyl or butyl acrylate or methacrylate,
vinyl or allylic esters of ethylenically unsaturated acids, for instance vinyl or allyl acetate or propionate,
vinyl or vinylidene halides, for instance vinyl or vinylidene chloride,
ethylenically unsaturated nitriles, for instance acrylonitrile,
hydroxyalkyl esters of ethylenically unsaturated acids, for instance hydroxyethyl or hydroxypropyl acrylate or methacrylate,
ethylenically unsaturated amides, for instance acrylamide or methacrylamide.

According to one advantageous embodiment, the polycarboxylate comprising hydrophobic units is a copolymer derived from maleic anhydride or from a maleic anhydride salt and from diisobutylene. Such a polymer is sold, for example, by the company Rhodia under the name Geropon® EGPM (as a solution, in sodium carboxylate form).

It is noted that the copolymers may be obtained in a manner known to those skilled in the art, for example by free-radical polymerization.

### Non-Polyalkoxylated Surfactant

The non-polyalkoxylated surfactant may be a nonionic non-polyalkoxylated surfactant, an anionic non-polyalkoxylated surfactant or an amphoteric non-polyalkoxylated surfactant, or a mixture of these surfactants.

Examples of amphoteric non-polyalkoxylated surfactants that may be mentioned include amphoacetates, amphodiacetates, betaines (carboxybetaines, for instance alkylbetaines and alkylamidoalkylbetaines), amine oxides and sultaines (sulfobetaines), and mixtures thereof.

Examples of non-polyalkoxylated surfactants that may be mentioned include alkyl sulfates, alkyl sulfonates, alkylaryl sulfonates and alkyl phosphates, and mixtures thereof.

Examples of nonionic non-polyalkoxylated surfactants that may be mentioned include the following surfactants:
alkylpolyglucosides,
non-polyalkoxylated sorbitan esters,
fatty acids,
mixtures thereof.

The dried emulsion may be a dried emulsion comprising as sole non-polyalkoxylated surfactant a sorbitan ester such as the surfactant Alkamuls S80 sold by Rhodia. It may, however, be a dried emulsion also comprising another surfactant. It may especially be different than a dried emulsion comprising as sole non-polyalkoxylated surfactant a sorbitan ester such as the surfactant Alkamuls S80 sold by Rhodia. The dried emulsion may be a dried emulsion comprising as sole non-polyalkoxylated surfactant a sorbitan ester such as the surfactant Alkamuls S80 sold by Rhodia and, as active-material solvent, a xylene-based solvent such as Solvesso 200 or 200 ND or tri-n-butyl phosphate (TBP), or a mixture of these solvents. It may, however, be different than a dried emulsion comprising as sole non-polyalkoxylated surfactant a sorbitan ester such as the surfactant Alkamuls S80 sold by Rhodia and, as active-material solvent, a xylene-based solvent such as Solvesso 200 or 200 ND or tri-n-butylphosphate (TBP), or a mixture of these solvents.

According to one advantageous embodiment, the non-polyalkoxylated surfactant is:
a sorbitan ester derived from a C₈-C₃₀ and preferably C₁₆-C₂₂ fatty acid, or
a mixture of a sorbitan ester derived from a C₈-C₃₀ and preferably C₁₆-C₂₂ fatty acid and of a C₈-C₃₀ and preferably C₁₆-C₂₂ fatty acid, or a salt of the fatty acid.

Preferably, the mixture of the sorbitan ester and of the fatty acid comprises, as solids, from 2% to 20% by weight of the fatty acid or of the salt.

The mixture of the sorbitan ester and of the fatty acid is a surfactant (surfactant composition or surfactant “blend”) that makes it possible to control the emulsification of hydrophobic phases, especially the size of the droplets of an emulsion comprising a hydrophobic phase dispersed in an aqueous phase. Thus, the invention also relates to this surfactant (this surfactant composition or surfactant “blend”) per se, especially a mixture comprising the sorbitan ester and the fatty acid or its salt, where appropriate with water, at a solids concentration of greater than 20% or 30% or 50% or 75%. The invention also relates to emulsions, which may or may not be dried, comprising this surfactant (and the hydrophobic phase and the aqueous phase), or to the use of the surfactant in emulsions.

### Amounts of Ingredients

Advantageously, the weight ratio between the hydrophobic phase and the matrix is between 50% and 80%.

Advantageously, the weight ratio between the non-polyalkoxylated surfactant and the hydrophobic phase is between 0.003 and 0.3 and preferably between 0.01 and 0.06.

Advantageously, the weight ratio between the non-polyalkoxylated surfactant and the matrix is between 0.006 and 0.6 and preferably between 0.02 and 1.2.

Advantageously, the matrix comprises at least 90% by weight of the water-soluble or water-dispersible polymer.

### Other Compounds

The dried emulsion may comprise other ingredients, which may be useful during its preparation, or which may be useful for modifying its properties or uses.

They may especially be active ingredients (i.e. ingredients having a function during the use, for example in a liquid formulation) included in the matrix, and formulable in an aqueous phase.

They may also be antifoams, saccharides as described in document WO 03/055584 (R 01186), or complexing agents comprising at least one of the elements from columns IIA, IVA, VA, VIII, IB and IIIB, making it possible more readily to control the release of the hydrophobic phase during redispersion in water, as described in document WO 03/006148 (R 01103).

The dried emulsion may optionally contain residual water. The water content is advantageously less than 10% by weight and even more preferably less than 3%.

### Process

The dried emulsion according to the invention may be prepared according to a process comprising the following steps:

a) preparing an emulsion comprising an aqueous phase in which is dispersed the hydrophobic phase dispersed in liquid form in water, the emulsion comprising the water-soluble or water-dispersible copolymer of the matrix the non-polyalkoxylated surfactant, where appropriate by heating the hydrophobic phase above its melting point,
b) removing the water, to obtain a dried emulsion,
c) optionally forming the dried emulsion into powder or granules,
d) recovering the dried emulsion.

### Step a) Preparation of the Emulsion

During a first step of the process, an emulsion is prepared comprising the hydrophobic phase, dispersed in the aqueous phase. The hydrophobic phase, during this step, is in liquid form. If need be, it is heated for this purpose. The emulsion comprises the water-soluble or water-dispersible polymer, and the surfactant.

Any method for preparing an emulsion may be used. These methods are known to those skilled in the art. Methods are described, for example, in the “Encyclopedia of Emulsions Technology”, volumes 1 to 3 by Paul Becher, published by Marcel Dekker Inc., 1983, and may be used in the context of the present invention.

Thus, the direct-phase emulsification method may be used. It is briefly recalled that this method consists in preparing a mixture containing the water and emulsifiers, including the water-soluble or water-dispersible polymer, and then in introducing the hydrophobic phase in liquid form, with stirring.

Another suitable method is phase-inversion emulsification. According to this route, the hydrophobic phase is mixed with an emulsifier, and the water, possibly containing the other constituents, for instance the water-soluble or water-dispersible polymer, is introduced dropwise with stirring. At and above a certain amount of introduced water, inversion of the emulsion takes place. An oil-in-water direct emulsion is then obtained. The emulsion obtained is then diluted in water so as to obtain a suitable volume fraction in dispersed phase.

Finally, the emulsion may be prepared by using colloidal mills such as Manton Gaulin and Microfluidizer mills (Microfluidics).

The mean size of the droplets of hydrophobic phase dispersed in the aqueous phase is generally between 0.1 μm and 50 μm, often between 1 and 10 micrometers and preferentially between 0.2 and 5 micrometers (expressed relative to the volume of particles; measured using a Horiba laser scattering granulometer).

The emulsification may be performed at a temperature in the region of room temperature, although lower or higher temperatures may be envisioned.

The amount of water present in the emulsion, before drying, may be between 5% and 99% by weight and preferably between 20% and 70% by weight. In general, small amounts of water are preferably used, since it must be removed thereafter. Controlling the amount of water may be a means of managing the viscosity.

### Steps b), c) and d): Drying, Forming

The method used for removing the water from the emulsion and obtaining the dried emulsion may be performed by any means known to those skilled in the art.

This operation takes place such that the various constituent components of the mixture are subjected to temperatures below their degradation temperatures.

According to a first embodiment of the invention, oven drying may be envisioned. Preferably, this drying takes place in a thin layer. More particularly, the temperature at which the drying is performed is less than or equal to 100° C., preferably between 30° C. and 90° C. and preferably between 50° C. and 90° C.

According to another particular embodiment of the invention, rapid drying of the mixture (or of the emulsion) is performed. Spray-drying, in a fluidized bed, using Duprat® drums, or freeze-drying (freezing-sublimation) is suitable in this respect.

Spray-drying, for example using a Niro machine, or in a fluidized bed, for example using an Aeromatic machine, may be performed in the usual manner in any known machine, for instance a spraying tower combining spraying performed with a nozzle or a turbine with a stream of hot gas. The inlet temperature of the hot gas (generally air), at the top of the column, is preferably between 50° C. and 250° C. and the outlet temperature is preferably less than the degradation temperature of the constituent components of the granule obtained.

In the case of operations for drying the mixture (or the emulsion) performed using a Duprat® drum, or any means for rapidly obtaining a dry film that is separated from the drying support by scraping, for example, particles that may optionally be ground are obtained. If necessary, these particles may be subjected to subsequent forming, for instance an agglomeration step, so as to obtain granules.

It should be noted that additives, such as anticaking agents, may be incorporated into the granules during this drying step.

It is recommended, for example, to use of filler chosen especially from calcium carbonate, barium sulfate, kaolin, silica, bentonite, titanium oxide, talc, hydrated alumina and calcium sulfoaluminate.

Preferably, the drying is performed such that at least 90% by weight and preferably between 90% and 95% by weight of the outer aqueous phase is removed. The residual amount of water is preferably less than 3% by weight.

### Uses:

The dried emulsion may be used:
in plant-protection formulations,
in laundry care formulations, for example in laundry washing powders or tablets, for the formulation of softeners, the vectorization of fragrances, the formulation of antifoams, or of silicones, for example for deposition onto fibers,
in automatic dishwasher formulations, in the form of powders or tablets,
in cosmetic formulations,
in household care wipes,
in skincare wipes,
in babycare wipes,
in panty liners,
in makeup-removing wipes,
in bath salt formulations,
in formulations for building and/or public works materials, for example for formulation of cement setting retarders or accelerators,
in surface coating formulations, for example in paints,
for formulating silicone oils in solid form, for example antifoams.

In greater detail, the dried emulsion may be used in plant-protection formulations, the hydrophobic phase being a plant-protection active compound, or a mixture comprising a plant-protection active hydrophobic compound, dissolved or dispersed in a hydrophobic solvent. The dried emulsion may optionally be used with other plant-protection products (combination of plant-protection products), for example in combination with a fertilizer. The dried emulsion and the fertilizer may especially be mixed in the same reservoir with water.

Other details or advantages of the invention may emerge in the light of the examples hereinbelow, which are given with no limiting nature.

Dried emulsions whose hydrophobic phase is metolachlor are produced, by preparing an emulsion comprising metolachlor, a surfactant, water and the polymer (Geropon® EGPM sold by Rhodia), followed by drying. The compositions (ingredients and amounts) of the emulsions and of the dried emulsions are given in the tables hereinbelow.

### Procedure:

### a) Preparation of the Emulsion

the polymer is weighed out, optionally with an antifoam
stirring is started at 500 rpm using a frame paddle
all or part of the water is added
a mixture of metolachlor and of the surfactant is added
the resulting mixture is homogenized and acidified with 2N HCl (dropwise)
this mixture is stirred at 500 rpm for 15 minutes
where appropriate, the rest of the water is added
the mixture is stirred for 30 minutes
the particle size (Horiba) is measured as a function of time

### b) Drying

the product is dried on a plate in an oven at 75° C. overnight
it is coarsely ground
the particle size is measured during redispersion (1 g of powder in 50 ml of mains water, with an electromagnetic stirrer, at 500 rpm for 5 minutes at room temperature.

### EXAMPLE 1

### Comparative

| | | | Dry | |
|---|---|---|---|---|
| | Dry extract (%) | Mass (g) | g | % |
| Metolachlor | 100 | 16.4 | 16.4 | 63.7 |
| Soprophor TSP | 100 | 1.2 | 1.2 | 4.7 |
| 8, Rhodia⁽¹⁾ | | | | |
| Geropon EGPM, | 25.8 | 30.5 | 7.9 | 30.6 |
| Rhodia | | | | |
| 2N HCl (73 g/l) | 7.3 | 3.6 | 0.3 | 1.0 |
| Mains water | | 22.0 | | |
| (introduced in | | | | |
| a single | | | | |
| portion) | | | | |
| | TOTAL | 73.7 | 25.7 | 100 |

⁽¹⁾polyethoxylated tristyrylphenol

### Emulsion Before Drying

Metolachlor/dry: 63.7%
Dry extract: 34.9%
Surfactant/(Metolachlor+surfactant): 6.8%
Surfactant/polymer: 13.2%

The emulsion is coarse. It is not subsequently dried.

### EXAMPLE 2

### Comparative

| | | | Dry | |
|---|---|---|---|---|
| | Dry extract (%) | Mass (g) | g | % |
| Metolachlor | 100 | 16.4 | 16.4 | 65.5 |
| Soprophor TSP | 100 | 0.5 | 0.5 | 2.0 |
| 10, Rhodia⁽¹⁾ | | | | |
| Geropon EGPM, | 25.8 | 30.5 | 7.9 | 31.4 |
| Rhodia | | | | |
| 2N HCl (73 g/l) | 7.3 | 3.6 | 0.3 | 1.0 |
| Mains water | | 5.0 + 15.0 | | |
| (introduced in | | | | |
| two portions) | | | | |
| | TOTAL | 71.0 | 25.0 | 100 |

⁽¹⁾polyethoxylated tristyrylphenol

### Emulsion Before Drying

Dry extract: 34.9%
Surfactant/(Metolachlor+surfactant): 3%
Surfactant/(surfactant+dry polymer): 6.0%

### Particle Size Before Drying:

### D10=0.54 μm

### D50=1.30 μm

### D90=3.35 μm

(D90−D10)/D50=2.16

The emulsion is not subsequently dried.

### EXAMPLE 3

| | | | Dry | |
|---|---|---|---|---|
| | Dry extract (%) | Mass (g) | g | % |
| Metolachlor | 100 | 16.4 | 16.4 | 65.5 |
| Rhodasurf CET2, | 100 | 0.5 | 0.5 | 2.0 |
| Rhodia⁽²⁾ | | | | |
| Geropon EGPM, | 25.8 | 30.5 | 7.9 | 31.4 |
| Rhodia | | | | |
| 2N HCl (73 g/l) | 7.3 | 3.6 | 0.26 | 1.0 |
| Mains water | | 5.0 + 15.0 | | |
| (introduced in | | | | |
| two portions) | | | | |
| | TOTAL | 71.0 | 25.0 | 100 |

⁽²⁾sorbitan ester

### Emulsion Before Drying

Dry extract: 35.3%
Surfactant/(Metolachlor+surfactant): 3%
Surfactant/(surfactant+dry polymer): 6.0%

### Particle Size Before Drying:

D10=0.228 μm bipopulous 21.156 μm
D50=0.329 μm bipopulous 28.625 μm
D90=0.453 μm bipopulous 38.645 μm

(D90−D10)/D50=0.684 bipopulous 0.611

The emulsion is not subsequently dried.

### EXAMPLE 4

| | | | Dry | |
|---|---|---|---|---|
| | Dry extract (%) | Mass (g) | g | % |
| Metolachlor | 100 | 16.4 | 16.4 | 65.5 |
| Surfactant | 100 | 0.5 | 0.5 | 2.0 |
| mixture⁽³⁾ | | | | |
| Geropon EGPM, | 25.8 | 30.5 | 7.9 | 31.4 |
| Rhodia | | | | |
| 2N HCl (73 g/l) | 7.3 | 3.6 | 0.26 | 1.0 |
| Mains water | | 7.5 + 12.5 | | |
| (introduced in | | | | |
| two portions) | | | | |
| | TOTAL | 71.0 | 25.0 | 100 |

⁽³⁾mixture comprising a sorbitan ester of oleic acid and 15% by weight of oleic acid

### Emulsion Before Drying

Dry extract: 35.3%
Surfactant/(Metolachlor+surfactant): 3%
Surfactant/(surfactant+dry polymer): 6.0%

### Particle Size Before Drying:

### D10=0.233 μm

### D50=0.303 μm

### D90=0.42 μm

(D90−D10)/D50=0.617

The emulsion is not subsequently dried.

### EXAMPLE 5

### Comparative

| | | | Dry | |
|---|---|---|---|---|
| | Dry extract (%) | Mass (g) | g | % |
| Metolachlor | 100 | 32.5 | 32.5 | 65.0 |
| Surfactant⁽⁴⁾ | 100 | 1 | 1 | 2.0 |
| Geropon EGPM, | 24.7 | 61.7 | 15.3 | 30.5 |
| Rhodia | | | | |
| 2N HCl (73 g/l) | 7.3 | 17.3 | 1.3 | 2.5 |
| Mains water | | 6.7 + 18.3 | | |
| (introduced in | | | | |
| two portions) | | | | |
| | TOTAL | 137.5 | | 100 |

⁽⁴⁾oleic acid

### Emulsion Before Drying

dry extract: 36.4

The emulsion is coarse. It is not subsequently dried.

### EXAMPLE 6

| | | | Dry | |
|---|---|---|---|---|
| | Dry extract (%) | Mass (g) | g | % |
| Metolachlor | 100 | 16.4 | 16.4 | 65.5 |
| Surfactant | 100 | 0.5 | 0.5 | 2.0 |
| mixture⁽⁵⁾ | | | | |
| Geropon EGPM, | 25.8 | 30.5 | 7.9 | 31.4 |
| Rhodia | | | | |
| 2N HCl (73 g/l) | 7.3 | 3.6 | 0.26 | 1.0 |
| Mains water | | 7.5 + 12.5 | | |
| (introduced in | | | | |
| two portions) | | | | |
| | TOTAL | 71.0 | 25.0 | 100 |

⁽⁵⁾mixture comprising a sorbitan ester of oleic acid and 8% by weight of oleic acid

### Emulsion Before Drying

Dry extract: 35.3%
Surfactant/(Metolachlor+surfactant): 3%
Surfactant/(surfactant+dry polymer): 6.0%

### Particle Size Before Drying:

### D10=0.124 μm

### D50=0.168 μm

### D90=0.226 μm

(D90−D10)/D50=0.607

### Particle Size After Redispersion:

### D10=0.258 μm

### D50=0.396 μm

### D90=0.591 μm

(D90−D10)/D50=0.841

The particle sizes before drying and after redispersion are noteworthy.

1. A dried emulsion comprising a matrix which comprises a water-soluble or water-dispersible polymer, in which is dispersed a hydrophobic phase, wherein:
the dried emulsion comprises a non-polyalkoxylated surfactant,
the water-soluble or water-dispersible polymer included in the matrix is a polycarboxylate comprising hydrophobic units.

2. The dried emulsion as defined by claim 1, wherein the hydrophobic phase is a liquid hydrophobic phase, or a solid hydrophobic phase.

3. The dried emulsion as defined by claim 1, wherein the hydrophobic phase comprises an active hydrophobic compound, or a mixture comprising an active hydrophobic compound dissolved or dispersed in a hydrophobic solvent.

4. The dried emulsion as defined by claim 1, wherein the polycarboxylate comprising hydrophobic units is a copolymer comprising units derived from the following monomers:
(I) monocarboxylic or polycarboxylic acid, or linear or branched, ethylenically unsaturated aliphatic, cyclic or aromatic anhydride,
(II) monomer of formula (R²)(R²)C═CH(R³) in which
R³ is a hydrogen atom or a methyl group, and
R², which may be identical or different, represent a hydrogen atom or a linear or branched, optionally cyclic, C₂-C₁₀ saturated or ethylenically unsaturated aliphatic or aromatic radical, with the proviso that the two radicals R² are not hydrogen atoms, and
optionally (III) polyoxyalkylenated ester of an ethylenically unsaturated carboxylic acid.

5. The dried emulsion as defined by claim 4, wherein the monomer (I) has the formula:
(R³)HC═C(R¹)—COOX (Ia)
in which:
R³ is a hydrogen atom or a methyl group,
R¹ is a hydrogen atom, a C₁-C₁₀ hydrocarbon-based radical optionally comprising a group —COOX, or a group —COOX, optionally forming with the group —COOX an anhydride group —CO—O—OC—, and
X is a hydrogen atom or a cation.

6. The dried emulsion as defined by claim 5, wherein the monomer (I) is selected from the group consisting of acrylic, methacrylic, citraconic, maleic, fumaric, itaconic and crotonic acid or anhydride.

7. The dried emulsion as defined by claim 4, wherein the monomer (II) is selected from the group consisting of 1-butene, isobutylene, n-1-pentene, 2-methyl-1-butene, n-1-hexene, 2-methyl-1-pentene, 4-methyl-1 pentene, 2-ethyl-1-butene, diisobutylene and 2-methyl-3,3-dimethyl-1-pentene.

8. The dried emulsion as defined by claim 1, wherein the polycarboxylate comprising hydrophobic units is a copolymer derived from maleic anhydride or from a maleic anhydride salt and from diisobutylene.

9. The dried emulsion as defined by claim 1, wherein the non-polyalkoxylated surfactant is a nonionic non-polyalkoxylated surfactant, an anionic non-polyalkoxylated surfactant or an amphoteric non-polyalkoxylated surfactant, or a mixture of these surfactants.

10. The dried emulsion as defined by claim 9, wherein the non-polyalkoxylated surfactant is selected from the group consisting of the following surfactants:
alkylpolyglucosides,
non-polyalkoxylated sorbitan esters,
fatty acids,
mixtures thereof.

11. The dried emulsion as defined by claim 9, wherein the non-polyalkoxylated surfactant comprises:
a sorbitan ester derived from a C₈-C₃₀ fatty acid, or
a mixture of a sorbitan ester derived from a C₈-C₃₀ fatty acid and of a C₈-C₃₀ fatty acid, or a salt of the fatty acid.

12. The dried emulsion as defined by claim 11, wherein the mixture of the sorbitan ester and of the fatty acid comprises, as solids, from 2% to 20% by weight of the fatty acid or of the salt.

13. The dried emulsion as defined by claim 1, wherein the weight ratio between the hydrophobic phase and the matrix is between 50% and 80%.

14. The dried emulsion as defined by claim 1, wherein the weight ratio between the non-polyalkoxylated surfactant and the hydrophobic phase is between 0.003% and 0.3.

15. The dried emulsion as defined by claim 1, wherein the weight ratio between the non-polyalkoxylated surfactant and the matrix is between 0.006 and 0.6.

16. The dried emulsion as defined by claim 1, wherein the matrix comprises at least 90% by weight of the water-soluble or water-dispersible polymer.

17. A process for preparing a dried emulsion as defined by claim 1, comprising the following steps:
a) preparing an emulsion comprising an aqueous phase in which is dispersed the hydrophobic phase dispersed in liquid form in water, the emulsion comprising the water-soluble or water-dispersible copolymer of the matrix and the non-polyalkoxylated surfactant, where appropriate by heating the hydrophobic phase above its melting point,
b) removing the water, to obtain a dried emulsion,
c) optionally forming the dried emulsion into powder or granules,
d) recovering the dried emulsion.

18. The process as defined by claim 17, wherein, during step b), the water is removed by thin-film evaporation, freeze-drying or by spraying of the emulsion.

19. The process as defined by claim 17, wherein the weight proportion between the aqueous phase and the hydrophobic phase is between 5% and 99%.

20.-22. (canceled)

23. A plant protection, laundry care, dishwasher or cosmetic formulation, household case, skincare or baby care wipes, panty liners, formulation for building and/or public works materials, or surface coating formulation, comprising the dried emulsion as defined by claim 1.

24. A plant protection formulation as defined by claim 23, the hydrophobic phase which comprises a plant protection active compound, or a mixture comprising a plant protection active hydrophobic compound, dissolved or dispersed in a hydrophobic solvent, the dried emulsion optionally being in combination with a fertilizer.

25. The plant protection formulation as defined by claim 24, in combination with a fertilizer, the dried emulsion and the fertilizer being mixed in the same reservoir with water.
